(19) United States
(12) Patent Application Publication
(10) Pub. No.: US 2023/0020109 A1
(43) Pub. Date: Jan. 19, 2023

(54) PHARMACEUTICAL COMPOSITION FOR ALLEVIATING EYE FATIGUE, CONTAINING, AS ACTIVE INGREDIENTS, LUTEOLIN-7-O-DIGLUCURONIDE AND APIGENIN-7-O-DIGLUCURONIDE ISOLATED FROM PERILLA FRUTESCENS (L.) BRITTON VAR. ACUTA (THUNB.) KUDO LEAF EXTRACT

(71) Applicant: JEONNAM BIOINDUSTRY FOUNDATION, Naju-si (KR)

(72) Inventors: Chul Yung CHOI, Gwangju (KR); Jae Yong KIM, Suncheon-si (KR); Hu Won KANG, Gwangju (KR); Ara JO, Gwangju (KR); Dool-Ri OH, Jeollanam-do (KR); Yu Jin KIM, Jeollanam-do (KR); So Jeong IM, Gwangju (KR); Seul Gi LEE, Suncheon-si (KR); Gyu Ok LEE, Jeollanam-do (KR); Sung Yoon PARK, Jeollanam-do (KR); Geun Chang RYU, Gwangju (KR); Rack Seon SEONG, Jeollanam-do (KR)

(21) Appl. No.: 17/942,958

(22) Filed: Sep. 12, 2022

Related U.S. Application Data
(62) Division of application No. 17/255,604, filed on Dec. 23, 2020.

(30) Foreign Application Priority Data
Jul. 18, 2018 (KR) 10-2018-0083438

(51) Int. Cl. A61K 36/535; A61P 27/02; A61K 31/7048

(52) U.S. Cl. CPC A61K 36/535; A61P 27/02; A61K 31/7048; A61K 2236/331; A61K 2236/51

(57) ABSTRACT

A pharmaceutical composition and a health functional food composition for alleviating eye fatigue containing, as active ingredients, compounds, particularly, luteolin-7-O-diglucuronide and apigenin-7-O-diglucuronide isolated from a Perilla frutescens (L.) Britton var. acuta (Thunb.) Kudo leaf extract have an effect of alleviating eye fatigue by increasing the contents of NO and cGMP associated with relaxation of ciliary smooth muscle cells isolated from rat's eyes and decreasing the content of [Ca²⁺]i, and increasing cGMP which is a factor associated with relaxation of the ciliary smooth muscle through an animal experiment. As a result, the luteolin-7-O-diglucuronide and apigenin-7-O-diglucuronide compounds extracted from Perilla frutescens (L.) Britton var. acuta (Thunb.) Kudo may be usefully used as a pharmaceutical composition and a health functional food composition having an eye fatigue alleviating effect as a Korean natural resource.

### TECHNICAL FIELD

The present invention relates to a pharmaceutical composition and a health functional food composition for alleviating eye fatigue containing, as active ingredients, luteolin-7-O-diglucuronide and apigenin-7-O-diglucuronide isolated from a Perilla frutescens (L.) Britton var. acuta (Thunb.) Kudo leaf extract.

### BACKGROUND ART

Perilla frutescens (L.) Britton var. acuta (Thunb.) Kudo is an annual medicinal plant belonging to the Lamiaceae genus, anthohyta family, and angiosperm class according to a biological classification and distributed in Korea and China.

The stems are upright and square, the whole tree is purple and has a scent, and leaves are symmetry, broad ovate, pointed at the end, rounded or somewhat wedge-shaped at the bottom, and serrated at the edge, and have hairs on both sides, especially long hairs on the veins and long petioles. Flowers bloom in light purple in August to September, and hang as a raceme on the ends of stems and branches, and upper leaf axils. The calyx is split into two parts, wherein the upper part is again in three rows and the lower part is in two rows, and has hairs inside and outside the tube. The corolla is a usually short labiate and has a lower sprout slightly longer than an upper sprout, the stamens are didynamous, and the fruit is a branch, round, and contained in the calyx. The pericarp is purple throughout, and the mericarp is circular and has a net pattern. Leaves and stems are used for medicinal purposes, and young leaves and seeds are edible.

Young leaves are similar enough to be hardly distinguished from perilla leaves, and sugar made from perylaldehyde contained in Perilla frutescens (L.) Britton var. acuta (Thunb.) Kudo is a sweetener 2,000 times stronger than normal sugar to be used for cigarettes, intestines, and toothpaste. Leaves that are green rather than purplish are called for.viridis. The for.viridis has white flowers, stronger scent than Perilla frutescens (L.) Britton var. acuta (Thunb.) Kudo, and is often used as medicinal materials.

On the other hand, the eyeball that recognizes objects is formed of a membrane-like tissue that is present in the innermost layer of the eye and plays an important role for visual functions such as light reception, and the retina is formed of 10 layers, for example, classified into a retinal pigment epithelium layer, a neuroepithelial layer, an outer boundary layer, a nuclear layer, an outer reticular layer, an inner granule layer, an inner reticular layer, a ganglion cell layer, a nerve fiber layer, and an inner boundary layer formed in the following order from the outside.

Light irradiated to the retina from the outside world is transmitted from the inner boundary layer side to the reticular layer and is received by visual cells (rod cells and cone cells) as photoreceptor cells existing in the neuroepithelial layer. In the visual cells, light is converted into neural signals, the signals are processed by various nerve cells in the retina, and information is finally transmitted to the cerebral center from ganglion cells on the surface of the retina through the optic nerve.

In the highly developed mechanical civilization, the eyes are easily fatigued due to various environmental pollution, excessive viewing of TV, excessive use of personal computers and electronic entertainment devices, etc., and when driving at night or working at night, a phenomenon of vision loss, such as a decrease in dark adaptability, occurs. In order to prevent such vision loss, various medicines, natural foods, etc. are favorably sought, and among them, a vision improving agent containing anthocyanosides extracted from wild blueberries as a main ingredient has been widely used at home and abroad.

In the present invention, the equivalence of luteolin-7-O-diglucuronide and apigenin-7-O-diglucuronide contained in freeze-dried and spray-dried powders of a Perilla frutescens (L.) Britton var. acuta (Thunb.) Kudo extract for the purpose of industrialization of Korean traditional plant resources was found. The present invention is to provide a pharmaceutical composition and a health functional food composition for alleviating eye fatigue containing active ingredients isolated from a Perilla frutescens (L.) Britton var. acuta (Thunb.) Kudo leaf extract.

### DISCLOSURE

### Technical Problem

An object of the present invention is to provide a pharmaceutical composition and a health functional food composition for alleviating eye fatigue containing, as active ingredients, compounds isolated from a Perilla frutescens (L.) Britton var. acuta (Thunb.) Kudo leaf extract which is a Korean natural resource.

### Technical Solution

In order to solve the problems, the present invention provides a pharmaceutical composition and a health functional food composition for alleviating eye fatigue containing, as active ingredients, compounds isolated from a Perilla frutescens (L.)Britton var. acuta (Thunb.) Kudo extract. The compounds isolated from the Perilla frutescens (L.) Britton var. acuta (Thunb.) Kudo leaf extract may be one or more flavonoid glycoside compounds selected from the group consisting of luteolin-7-O-diglucuronide and apigenin-7-O-diglucuronide, and the Perilla frutescens (L.) Britton var. acuta (Thunb.) Kudo leaf extract includes an extract soluble in any one of water, an alcohol having 1 to 5 carbon atoms, or a mixed solvent thereof.

The present invention provides a method for preparing a flavonoid glycoside compound comprising a process of identifying a structure of the extract by NMR and MS by isolating the fractions prepared by performing diaion HP-20 resin to prepare Perilla frutescens (L.) Britton var. acuta (Thunb.) Kudo leaf fractions.

The health functional food composition may be used as a pharmaceutical composition and a health function food composition for alleviating eye fatigue, having an eye fatigue alleviating effect, such as tablets, capsules, pills, granules, liquids, powders, flakes, paste, syrups, etc. according to a general method, respectively.

### Advantageous Effects

According to the present invention, it was confirmed that a pharmaceutical composition and a health functional food composition for alleviating eye fatigue containing, as active ingredients, compounds, particularly, luteolin-7-O-diglucuronide and apigenin-7-O-diglucuronide isolated from a Perilla frutescens (L.) Britton var. acuta (Thunb.) Kudo leaf extract have an effect of alleviating eye fatigue by increasing the contents of NO and cGMP associated with relaxation of ciliary smooth muscle cells isolated from rat's eyes and decreasing the content of [Ca²⁺]i, and increasing cGMP which is a factor associated with relaxation of the ciliary smooth muscle through an animal experiment. As a result, the luteolin-7-O-diglucuronide and apigenin-7-O-diglucuronide compounds extracted from Perilla frutescens (L.) Britton var. acuta (Thunb.) Kudo may be usefully used as a pharmaceutical composition and a health functional food composition having an eye fatigue alleviating effect as a Korean natural resource.

### BRIEF DESCRIPTION OF THE DRAWINGS

FIG. 1 illustrates compounds contained in a Perilla frutescens (L.) Britton var. acuta (Thunb.) Kudo leaf extract through HPLC analysis. (1) Luteolin-7-O-diglucuronide, (2) Apigenin-7-O-diglucuronide, (3) Rosmarinic acid

FIGS. 2A-2B illustrate an effect of inhibiting ROS production of a Perilla frutescens (L.) Britton var. acuta (Thunb.) Kudo leaf extract using C2C12 cells. FIG. 2A represents an MTT assay (cytotoxicity) result, and FIG. 2B represents an ROS measurement result.

FIG. 3 illustrates a relaxation effect of the Perilla frutescens (L.) Britton var. acuta (Thunb.) Kudo leaf extract in the ciliary muscle isolated from a rabbit.

FIGS. 4A-4B illustrate changes in contents of cGMP and cAMP of the Perilla frutescens (L.) Britton var. acuta (Thunb.) Kudo leaf extract in the aortic smooth muscle cells. FIG. 4A represents a cGMP content result for 15, 30 and 60 minutes, and FIG. 4B represents a cAMP content result for 15, 30 and 60 minutes.

FIGS. 5A-5B illustrate inhibition of PDE5A and PDE3A activities of the Perilla frutescens (L.) Britton var. acuta (Thunb.) Kudo leaf extract in the aortic smooth muscle cells, respectively.

FIGS. 6A-6B illustrate a change in [Ca2+]i concentration of the Perilla frutescens (L.) Britton var. acuta (Thunb.) Kudo leaf extract in the aortic smooth muscle cells.

FIG. 7 illustrates an effect of inhibiting the [Ca2+]i content induced by ET-1 in aortic smooth muscle cells of the Perilla frutescens (L.) Britton var. acuta (Thunb.) Kudo leaf extract.

FIG. 8 illustrates a schematic diagram of isolation of luteolin-7-O-diglucuronide and apigenin-7-O-diglucuronide compounds isolated from the Perilla frutescens (L.) Britton var. acuta (Thunb.) Kudo leaf extract.

FIG. 9 illustrates a result of luteolin-7-O-diglucuronide and apigenin-7-O-diglucuronide ingredients isolated from the Perilla frutescens (L.) Britton var. acuta (Thunb.) Kudo leaf extract. Here, (A-1): compounds structures of luteolin-7-O-diglucuronide, (A-2): HPLC analysis of luteolin-7-O-diglucuronide, (A-3): UV spectrum of luteolin-7-O-diglucuronide, (B-1): compounds structures of apigenin-7-O-diglucuronide, (B-2): HPLC analysis of apigenin-7-O-diglucuronide, and (B-3): UV spectrum of apigenin-7-O-diglucuronide.

FIG. 10 illustrates the equivalence of luteolin-7-O-diglucuronide and apigenin-7-diglucuronide compounds according to a freeze-drying and spray-drying method of the hot water extract of Perilla frutescens (L.) Britton var. acuta (Thunb.) Kudo leaves. Here, FD represents freeze-dried, and SD represents spray-dried.

FIGS. 11A-11B illustrate cytotoxicity and NO content of ciliary muscle cells (rCSMCs) isolated from rat's eyes (SD rat) for the freeze-dried product of the Perilla frutescens (L.) Britton var. acuta (Thunb.) Kudo leaf extract, respectively.

FIGS. 12A-12B illustrate changes in contents of cGMP and cAMP of ciliary muscle cells (rCSMCs) isolated from rat's eyes for the freeze-dried product of the Perilla frutescens (L.) Britton var. acuta (Thunb.) Kudo leaf extract, respectively.

FIGS. 13A-13B illustrate a change in ca2+ concentration of ciliary muscle cells (rCSMCs) isolated from rat's eyes for the Perilla frutescens (L.) Britton var. acuta (Thunb.) Kudo leaf extract. FIG. 13A illustrates an effect of the freeze-dried product of the Perilla frutescens (L.) Britton var. acuta (Thunb.) Kudo extract on the [Ca2+]i content of ciliary muscle cells isolated from an SD rat; and FIG. 13B illustrates an amount of change in [Ca2+]i content of ciliary muscle cells isolated from an SD rat in the freeze-dried product of the Perilla frutescens (L.) Britton var. acuta (Thunb.) Kudo extract.

FIG. 14 illustrates a result of measuring a change in cGMP content of rat's eyes after orally administering 100 and 200 mg/kg of the Perilla frutescens (L.) Britton var. acuta (Thunb.) Kudo leaf extract for 3 days after inducing the fatigue by irradiating light to the rat's eyes.

FIG. 15 illustrates changes in cGMP content of ciliary muscle cells (rCSMCs) isolated from rat's eyes for the Perilla frutescens (L.) Britton var. acuta (Thunb.) Kudo leaf extract.

### BEST MODE FOR THE INVENTION

A health functional food composition for alleviating eye fatigue of the present invention contains, as active ingredients, compounds isolated from a Perilla frutescens (L.) Britton var. acuta (Thunb.) Kudo leaf extract. In the following Examples of the present invention, as compounds isolated from the Perilla frutescens (L.) Britton var. acuta (Thunb.) Kudo leaf extract, luteolin-7-O-diglucuronide and apigenin-7-O-diglucuronide compounds will be described as examples. In order to optimize industrialization, differences in changes of the contents of luteolin-7-O-diglucuronide and apigenin-7-O-diglucuronide in the Perilla frutescens (L.) Britton var. acuta (Thunb.) Kudo extract were confirmed according to a drying method.

It was confirmed that the flavonoid compound increased the content of cGMP, which was a relaxation factor for the ciliary muscle of the eye, to relax the contracted ciliary muscle, thereby alleviating eye fatigue.

In the present invention, the flavonoid glycoside compound is at least one selected from the group consisting of luteolin-7-O-diglucuronide represented by the following Chemical Formula 1, and apenin-7-O-diglucuronide represented by the following Chemical Formula 2.

### EXAMPLE 1

### Effect of Perilla frutescens (L.) Britton var. acuta (Thunb.) Kudo Extract on Eye Fatigue in Aortic Smooth Muscle Cells (In Vitro), Ciliary Muscle (Ex Vivo) of Rabbit'S Eyes, and Near Point Accommodation (Human Application Test)

1. Preparation of Hot Water Extract of Perilla frutescens (L.) Britton var. acuta (Thunb.) Kudo Leaves

3 kg of Perilla frutescens (L.) Britton var. acuta (Thunb.) Kudo leaves (dried leaves) were extracted with hot water at 100° C. for 3 hours using 10-fold distilled water. The extracted water extract was concentrated under reduced pressure and freeze-dried to obtain 650 g of a hot water freeze-dried product of Perilla frutescens (L.) Britton var. acuta (Thunb.) Kudo leaves.

2. Component Analysis of Perilla frutescens (L.) Britton var. acuta (Thunb.) Kudo Extract Using HPLC

An HPLC device used for component analysis of a hot water extract of Perilla frutescens (L.) Britton var. acuta (Thunb.) Kudo leaves was an YL 9100 HPLC system, and the column used Triart C18 plus (250×4.6 mm, 5 um, YNC co. Ltd). A mobile phase was methanol (mobile phase A) and distilled water for HPLC (mobile phase B, 0.1% formic acid), and the ratio of methanol was adjusted from 30% (0 to 10 minutes) to 30% to 50% (10 to 30 minutes), 60% (35 to 40 minutes), 60% to 70% (40 to 45 minutes), 70% to 100% (45 to 53 minutes), and 100% (53 to 56 minutes) and finally adjusted to 30% (56 to 60 minutes), the flow rate was 1 mL/min, and analyzed at 325 nm using a UV/VIS (9120) detector.

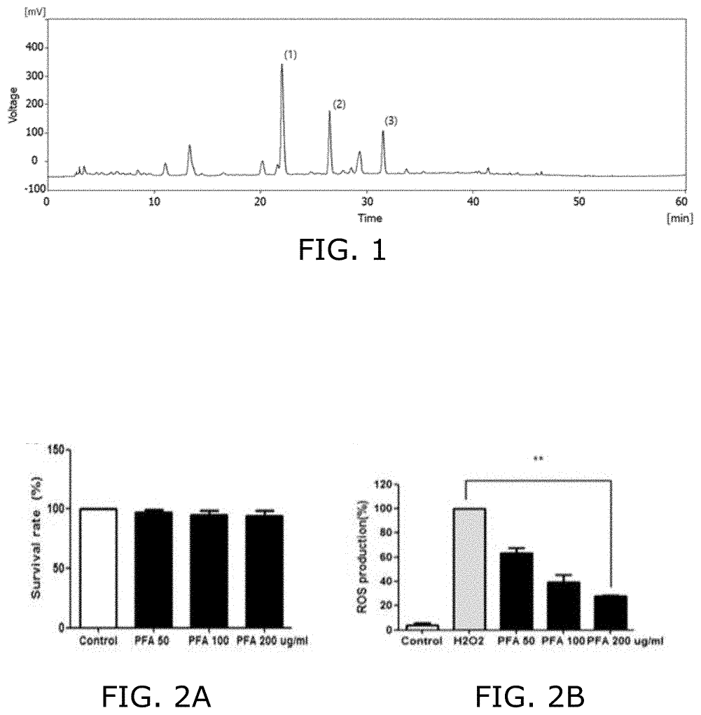

FIG. 1 illustrates compounds contained in a Perilla frutescens (L.) Britton var. acuta (Thunb.) Kudo leaf extract through HPLC analysis. As a result of analyzing the hot water extract of Perilla frutescens (L.) Britton var. acuta (Thunb.) Kudo leaves using HPLC, it was confirmed that main substances such as rosmarinic acid, luteolin-7-O-diglucuronide, and apigenin-7-O-diglucuronide compounds were contained. In the drawing, (1) represents luteolin-7-O-diglucuronide, (2) represents apigenin-7-O-diglucuronide, and (3) represents rosmarinic acid.

3. ROS Measurement Using C₂C₁₂ Cells

C₂C₁₂ cells were dispensed into a 48 well plate at 5×10⁵ cells/mL, treated with hydrogen peroxide (H₂O₂, 200 μM) for 2 hours to induce oxidative stress, and then reacted with the Perilla frutescens (L.) Britton var. acuta (Thunb.) Kudo extract (50, 100, and 200 μg/mL) for 24 hours. Cytotoxicity was measured through an MTT method. To measure ROS, the cells were washed twice with PBS by removing the medium, and then treated with 1% Triton X-100 (PBS) to lyse the cells at 37° C. for 10 minutes. DCF-DA (10 μM) reacted at room temperature in a darkroom for 30 minutes. After the reaction, the cells were washed twice with cold PBS, and measured at excitation 485 nm and emission 530 nm using a fluorescence spectrometer.

FIGS. 2A-2B illustrate an effect of inhibiting ROS production of a Perilla frutescens (L.) Britton var. acuta (Thunb.) Kudo leaf extract using C₂C₁₂ cells. FIG. 2A represents an MTT assay (cytotoxicity) result, and FIG. 2B represents an ROS measurement result. It was confirmed that the Perilla frutescens (L.) Britton var. acuta (Thunb.) Kudo leaf extract (50, 100, and 200 μg/mL) inhibited concentration-dependent production of ROS, which was an oxidative stress substance induced by H₂O₂, without toxicity of C₂C₁₂ cells.

4. Measurement of Relaxation Rate of Ciliary Muscle of Rabbit's Eye

A rabbit (2.4 to 2.7 kg) was purchased as an experimental animal and adapted to an environment to be used in this test. General anesthesia was performed by intramuscular injection of a zoletil-rompun mixture (1:2) to the experimental animal, and the eyeball was extracted. The extracted eyeball was cut in half at the equator of the eyeball by cornea incision, and the ciliary muscle was carefully isolated from the cornea after removing the eye lens. The isolated ciliary muscle was cut into a sample having a width of 3 mm×a length of 6 mm to obtain a sample.

Ciliary muscle slices were ventilated with mixed gas of 95% oxygen and 5% carbon dioxide, and added in Krebs-Henseleit (CaCl₂ 1.5 mM, NaCl 118 mM, KCl 4.7 nM, MgSO₄ 1.1 mM, KH₂PO₄ 1.2 mM, NaHCO₃ 25 mM, glucose 10 mM; pH 7.4) solution to perform the experiment. The contraction of the ciliary muscle was suspended by applying a load of 1 g using a tension transducer. The ciliary muscle slices were stabilized for 90 minutes. After stabilization, carbachol (100 μM/mL) was added to contract the ciliary muscle. It was confirmed that after the last stimulus, the Perilla frutescens (L.) Britton var. acuta (Thunb.) Kudo leaf extract (100 and 200 μg/mL) was added to the ciliary muscle to confirm the relaxation rate.

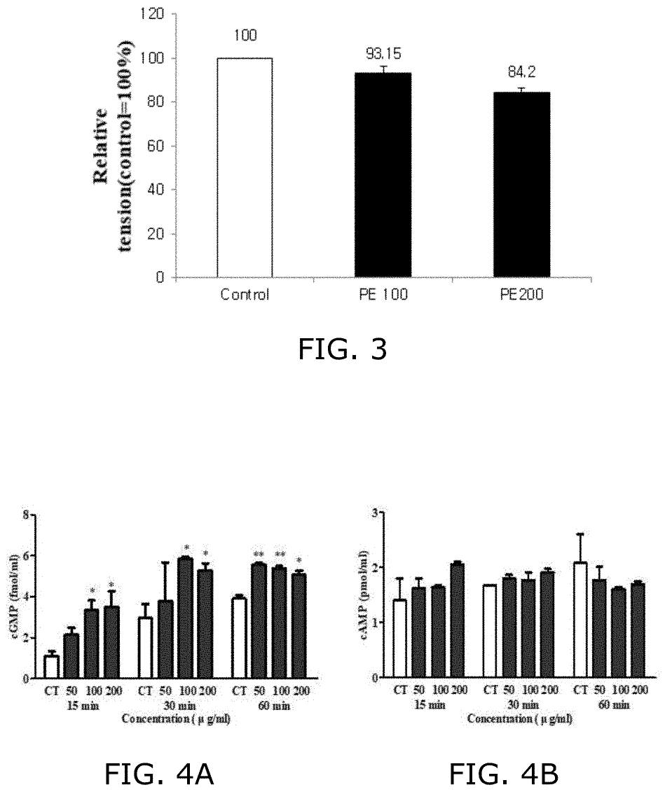

FIG. 3 illustrates a relaxation effect of the Perilla frutescens (L.) Britton var. acuta (Thunb.) Kudo leaf extract in the ciliary muscle isolated from a rabbit. It was confirmed whether the Perilla frutescens (L.) Britton var. acuta (Thunb.) Kudo leaf extract affected the relaxation of the ciliary muscle isolated from the rabbit. That is, after contracting the ciliary muscle of the rabbit using carbachol (100 μM/ml), 100 and 200 μg/mL of the Perilla frutescens (L.) Britton var. acuta (Thunb.) Kudo extract were added to confirm the relaxation rate. A group added with distilled water (control) did not affect the contracted ciliary muscle, whereas 200 μg/mL of the Perilla frutescens (L.) Britton var. acuta (Thunb.) Kudo extract significantly relaxed the ciliary muscle contracted by carbachol.

5. Relaxation Mechanism Using Aortic Smooth Muscle Cells

5.1. Measurement of Contents of cGMP and cAMP

Primary human aortic smooth muscle cells (hASMCs) were purchased from ATCC (American Type Culture Collection, PCS-100-012, Manassas, Va., USA), and incubated in a 5% CO₂ incubator in a vascular cell basal medium added with a vascular smooth muscle cell growth kit. hASMC was dispensed into 6 wells at 5×10⁵ cells/well and stabilized for 24 hours. After stabilization, 3-isobuytyl-1-methylxanthine (IBMX, 1 mM) was pre-treated for 10 minutes, and then a Perilla frutescens (L.) Britton var. acuta (Thunb.) Kudo leaf extract (50, 100, 200 μg/mL) was reacted for 15, 30, and 60 minutes. The contents of cGMP and cAMP of the Perilla frutescens (L.) Britton var. acuta (Thunb.) Kudo extract were measured using an ELISA kit.

FIGS. 4A-4B illustrate changes in contents of cGMP and cAMP of the Perilla frutescens (L.) Britton var. acuta (Thunb.) Kudo leaf extract in the aortic smooth muscle cells. FIG. 4A represents a cGMP content result for 15, 30 and 60 minutes, and FIG. 4B represents a cAMP content result for 15, 30 and 60 minutes. That is, it was confirmed that the Perilla frutescens (L.) Britton var. acuta (Thunb.) Kudo leaf extract (50, 100, 200 μg/mL) increased the cGMP content associated with relaxation of the ciliary muscle in a concentration-dependent manner, and increased the cGMP content for each reaction time (15 to 60 minutes) of the Perilla frutescens (L.) Britton var. acuta (Thunb.) Kudo leaf extract. On the other hand, the Perilla frutescens (L.) Britton var. acuta (Thunb.) Kudo leaf extract did not affect a change in content of cAMP.

5.2. Measurement of Phosphodiesterase (PDE) Inhibition Rate

PDE5A and PDE3A activities were measured according to a method of a kit (BPS Bioscience, San Diego, Calif.). That is, 50 μL of a reaction mixture (PDE5A 10 ng/ml, PDE3A 20 ng/ml, FAM-Cyclic-3′, 5′-GMP, FAM-Cyclic-3′, 5′-AMP 200 nM) was added and the Perilla frutescens (L.) Britton var. acuta (Thunb.) Kudo leaf extract (50, 100, 200 μg/mL) was added. The reaction mixture was reacted at room temperature for 1 hour. A diluted binding agent (100 μL) was added and the reaction mixture was reacted for 1 hour again. Fluorescence polarization of each sample was measured at excitation 480 nm and emission 528 nm.

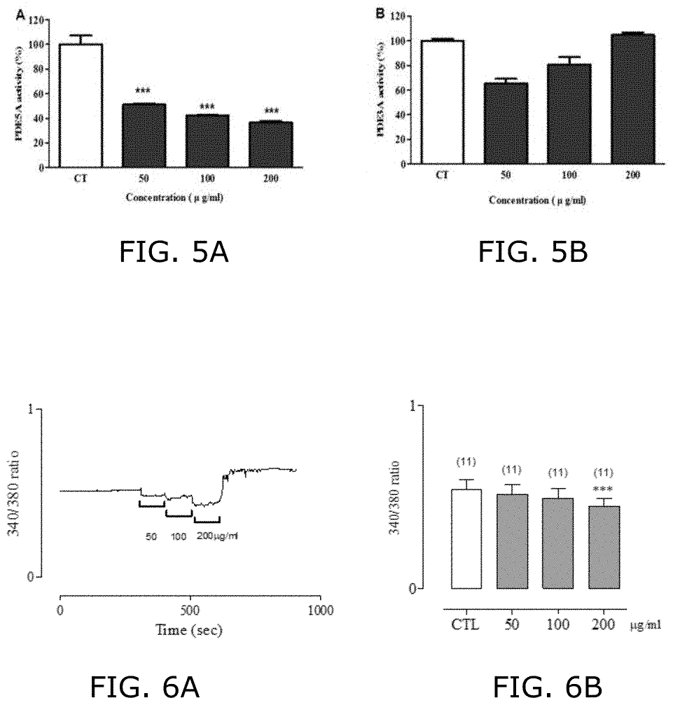

FIGS. 5A-5B illustrate inhibition of PDE5A and PDE3A activities of the Perilla frutescens (L.) Britton var. acuta (Thunb.) Kudo leaf extract in the aortic smooth muscle cells, respectively. FIG. 5A represents the PDE5A activity result, and FIG. 5B represents the PDE3A activity result. The Perilla frutescens (L.) Britton var. acuta (Thunb.) Kudo leaf extract (50, 100, 200 μg/mL) inhibited the PDE5A activity in a concentration-dependent manner, but had no effect on inhibition of the PDE3A activity. That is, the PDE5A activity exhibited inhibition rates of 51.23±0.29%, 42.42±0.13% and 36.58±0.37% at 50, 100, and 200 μg/mL concentrations of the Perilla frutescens (L.) Britton var. acuta (Thunb.) Kudo extract, respectively. Therefore, it was found that the Perilla frutescens (L.) Britton var. acuta (Thunb.) Kudo extract affected the relaxation of the smooth muscle by a cGMP mechanism.

5.3. Measurement of [Ca2+]i Content

A calcium ion-sensitive fluorescent material, an acetoxymethyl-ester form, fura-2/AM (Molecular probes, Eugene, Oreg.) was used as a calcium ion labeling material. The cells were treated with 5 μM fura-2/AM and 0.001% F127 in a HEPES buffer while blocking the light, and reacted at room temperature for 60 minutes. The cells were washed several times with the HEPES buffer and flowed with the buffer for 5 minutes to be stabilized. Hot water extracts (50, 100 and 200 μg/mL) of Perilla frutescens (L.) Britton var. acuta (Thunb.) Kudo were sequentially treated for 100 seconds by concentration. After stabilizing the cells for 5 minutes in the same manner, the hot water extracts of Perilla frutescens (L.) Britton var. acuta (Thunb.) Kudo leaves were treated with one concentration for 100 seconds.

In addition, the cells were treated with endothelin-1 (ET-1; 10 nM) and stimulated for 100 seconds, and treated with the hot water extracts of Perilla frutescens (L.) Britton var. acuta (Thunb.) Kudo (50, 100 and 200 μg/mL) for 100 seconds by concentration. At this time, all buffers and chemicals were treated by a perfusion device by gravity. Light from a lamp was selectively exposed to the cells with wavelengths of 340 nm and 380 nm through a computer control wheel. Photos were taken at 340 nm and 380 nm every 2 seconds, and emitter fluorescence light passing through a 515 nm long-pass filter passed through a cooled CCD camera and obtained a ratio value of 340 nm/380 nm by a digital fluorescence analyzer.

FIGS. 6A-6B illustrate a change in [Ca²⁺]i concentration of the Perilla frutescens (L.) Britton var. acuta (Thunb.) Kudo leaf extract in the aortic smooth muscle cells. That is, the Perilla frutescens (L.) Britton var. acuta (Thunb.) Kudo leaf extract (50, 100, 200 μg/mL) of aortic smooth muscle cells (rCSMCs) for the Perilla frutescens (L.) Britton var. acuta (Thunb.) Kudo leaf extract decreased the [Ca²⁺]i concentration content associated with contraction of the ciliary muscle in a concentration-dependent manner.

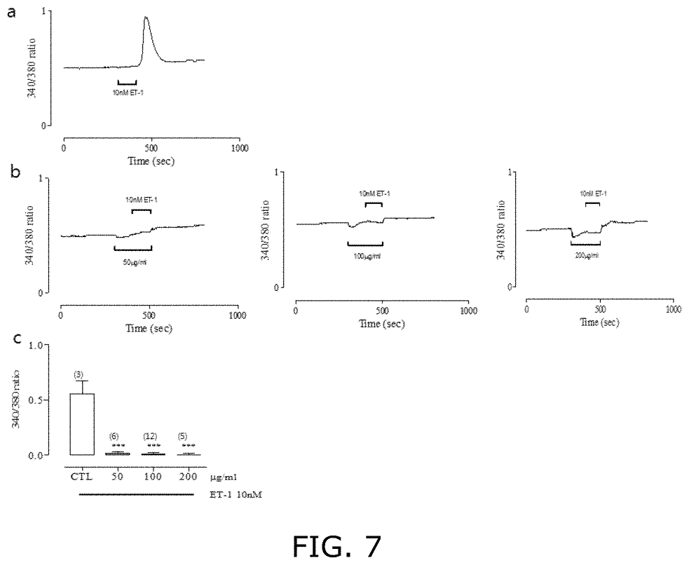

FIG. 7 illustrates an effect of inhibiting the [Ca2+]i content induced by ET-1 in aortic smooth muscle cells of the Perilla frutescens (L.) Britton var. acuta (Thunb.) Kudo leaf extract. After increasing the concentration content of [Ca2+]i by adding ET-1 (10 uM) to hASMCs cells, the Perilla frutescens (L.) Britton var. acuta (Thunb.) Kudo leaf extract was added with 50, 100, and 200 μg/mL to measure the concentration content of [Ca2+]i. As a result, the Perilla frutescens (L.) Britton var. acuta (Thunb.) Kudo leaf extract significantly decreased the concentration of [Ca2+]i.

### EXAMPLE 2

### Eye Fatigue Effect of luteolin-7-O-diglucuronide and apigenin-7-O-diglucuronide Contained in Perilla frutescens (L.) Britton var. acuta (Thunb.) Kudo Extract through Ciliary Muscle Cells Isolated from SD Rat

1. Preparation of Hot Water Extract of Perilla frutescens (L.) Britton var. acuta (Thunb.) Kudo Leaves and Isolation of Flavonoid Glycoside Compound

1-1. Preparation of Water Extract of Perilla frutescens (L.) Britton var. acuta (Thunb.) Kudo Leaves

3 kg of Perilla frutescens (L.) Britton var. acuta (Thunb.) Kudo dried leaves were extracted with hot water at 100° C. for 3 hours using distilled water. The extract was concentrated under reduced pressure and then freeze-dried and spray-dried to obtain a freeze-dried product (650 g, 21.6%) and a spray-dried product (669 g, 22.4%) of the hot water extract of the Perilla frutescens (L.) Britton var. acuta (Thunb.) Kudo leaves.

1-2. Isolation of Active Ingredients Using Diaion HP-20 Resin

In order to isolate active ingredients from a hot water extract of the Perilla frutescens (L.) Britton var. acuta (Thunb.) Kudo leaves, 2 L of the hot water extract of the Perilla frutescens (L.) Britton var. acuta (Thunb.) Kudo leaves was added to Diaion HP-20 resin and water and methanol were sequentially eluted in ratios of 30:70, 50:50, 70:30, and 0:100. Finally, 2 L of acetone was eluted to obtain 5 fractions. Of the five fractions, a first small fraction (30:70) was obtained using preparative Waters HPLC as compound 1 (150 mg, purity 96.7%) and compound 2 (50 mg, purity 96.4%).

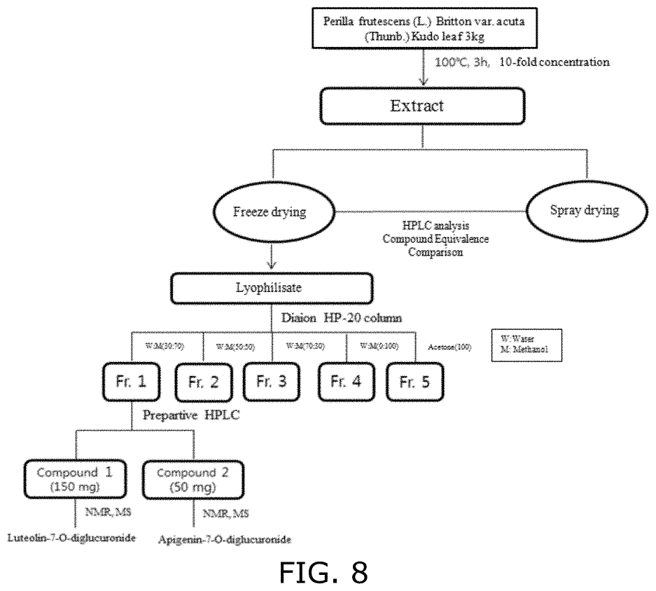

FIG. 8 illustrates a schematic diagram of isolation of luteolin-7-O-diglucuronide and apigenin-7-O-diglucuronide compounds isolated from the Perilla frutescens (L.) Britton var. acuta (Thunb.) Kudo leaf extract.

1-3. Structure Identification Using NMR

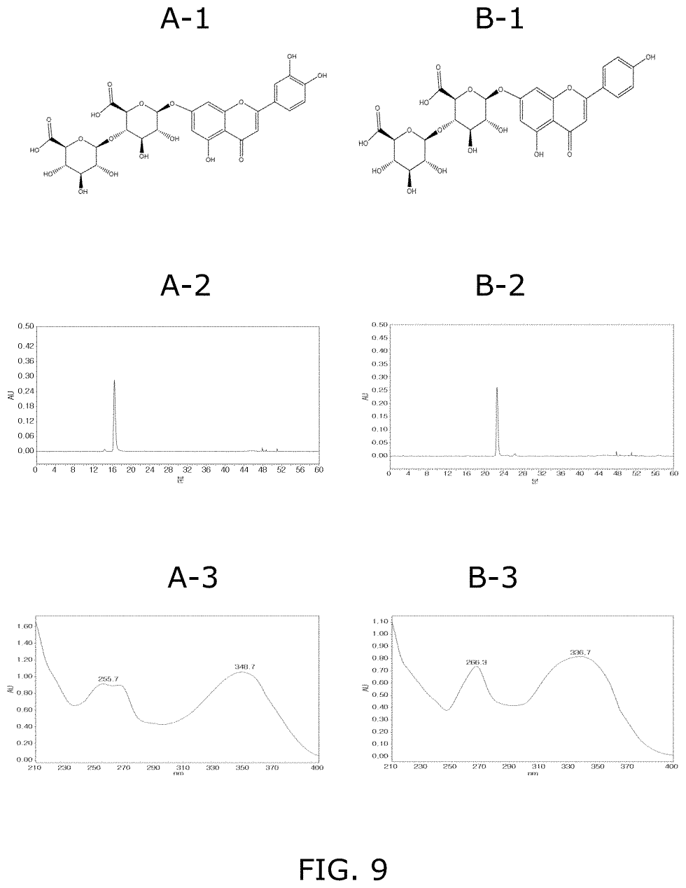

The structures of the substances obtained in Example 1-2, compound 1 and compound 2, were identified as luteolin-7-O-diglucuronide and apenin-7-O-diglucuronide using NMR and MS, respectively. FIG. 9 illustrates a result of luteolin-7-O-diglucuronide and apigenin-7-O-diglucuronide ingredients isolated from the Perilla frutescens (L.) Britton var. acuta (Thunb.) Kudo leaf extract. Here, (A-1): compounds structures of luteolin-7-O-diglucuronide, (A-2): HPLC analysis of luteolin-7-O-diglucuronide, (A-3): UV spectrum of luteolin-7-O-diglucuronide, (B-1): compounds structures of apigenin-7-O-diglucuronide, (B-2): HPLC analysis of apigenin-7-O-diglucuronide, and (B-3): UV spectrum of apigenin-7-O-diglucuronide.

The analysis structures of NMR and MS of luteolin-7-O-diglucuronide and apenin-7-O-diglucuronide identified, respectively, are as follows.

1) Analysis Structures of luteolin-7-O-diglucuronide

Compound 1, buff powder, ESI-MS: 639 (M+H)⁺. ¹HNMR (500 MHz, pyridine-d5) δ 4.25˜4.74 (1H, H-1 GluA2), 4.38˜4.76 (1H, d, J=7.2 Hz, H-1 GluA1), 4.92 (1H, d, J=9.5 Hz, H-5″), 5.57 (1H, d, J=8 Hz, H-1′″), 6.05 (1H, d, J=7 Hz, H-1″), 6.82 (1H, s, H-3), 7.13 (1H, d, J=2 Hz, H-6), 7.17(1H, d, J=2 Hz, H-8), 7.21 (1H, d, J=8.5 Hz, H-5′), 7.43 (1H, dd, J=2.0, 8.5 Hz, H-6′), 7.85 (1H, d, J=2 Hz, H-2′).

¹³CNMR (125 MHz, pyridine-d5) δ 72.43 (C-4″), 73.16 (C-4′″), 76.02(C-2′″), 76.80(C-5″), 77.40 (C-3″), 77.62 (C-3′″), 78.06 (C-5′″), 83.89 (C-2″), 95.61 (C-8), 100.06 (C-1″), 100.64 (C-6), 103.77 (C-3), 106.57 (C-1′″), 106.75 (C-10), 114.49 (C-2′), 116.57 (C-5), 119.47 (C-6′), 122.46 (C-1), 147.47(C-3′), 151.61(C-4′), 157.55(C-5), 162.46 (C-9), 163.47 (C-7), 165.00 (C-2), 171.79 (C-6″), 172.37 (C-6′″), 182.61 (C-4).

2) Analysis Structures of apenin-7-O-diglucuronide

Compound 2, buff powder, ESI-MS: 623 (M+H)⁺. ¹HNMR (500 MHz, pyridine-d5) δ 4.25˜4.65 (1H, H-1 GluA2), 4.38˜4.77 (1H, d, J=7.2 Hz, H-1 GluA1), 4.95 (1H, d, J=7 Hz, H-5″), 5.58 (1H, d, J=8 Hz, H-1′″), 6.08 (1H, d, J=7 Hz, H-1″), 6.81 (1H, s, H-3), 7.13 (1H, d, J=2 Hz, H-6), 7.16 (1H, d, J=2 Hz, H-8), 7.20 (1H, d, J=8.5 Hz, H-5′), 7.29 (1H, dd, J=2.0, 8.5 Hz, H-6′), 7.80 (1H, d, J=8.5 Hz,

¹³CNMR (125 MHz, pyridine-d5): 72.46 (C-4″), 73.16 (C-4′″), 76.02(C-2′″), 76.83 (C-3″), 77.43 (C-5″), 77.63 (C-3′″), 78.06 (C-5′″), 83.95 (C-2″), 95.60 (C-8), 100.06 (C-1″), 100.78 (C-6), 103.69 (C-3), 106.57 (C-10), 106.79 (C-1′″), 116.57 (C-3), 116.57 (C-5), 121.82 (C-1), 128.74 (C-2′), 128.74 (C-6′), 157.56 (C-5), 162.46 (C-9), 162.52 (C-4′), 163.58 (C-7), 164.63 (C-2), 171.78 (C-6′″), 172.36 (C-6′″), 182.64 (C-4).

2. Comparison of Compound Equivalence According to Hot Water Extraction and Drying Method of Perilla frutescens (L.) Britton var. acuta (Thunb.) Kudo Leaves

The equivalence of the compounds of the freeze-dried product and the spray-dried product of the hot water extract of Perilla frutescens (L.) Britton var. acuta (Thunb.) Kudo leaves was analyzed by HPLC. The HPLC device used was a Waters series HPLC system (Waters corporation 34 Maple street Milford, Mass.), and a column was Triart C18 plus (250×4.6 mm, 5 um, YNC co. Ltd). A mobile phase was methanol (mobile phase A) and distilled water for HPLC (mobile phase B, 0.1% formic acid), and the ratio of methanol was adjusted from 30% (0 to 10 minutes) to 30% to 50% (10 to 30 minutes), 60% (35 to 40 minutes), 60% to 70% (40 to 45 minutes), 70% to 100% (45 to 53 minutes), and 100% (53 to 56 minutes) and finally adjusted to 30% (56 to 60 minutes), the flow rate was 1 mL/min, and analyzed at 254 nm using a photodiode array (2998) detector.

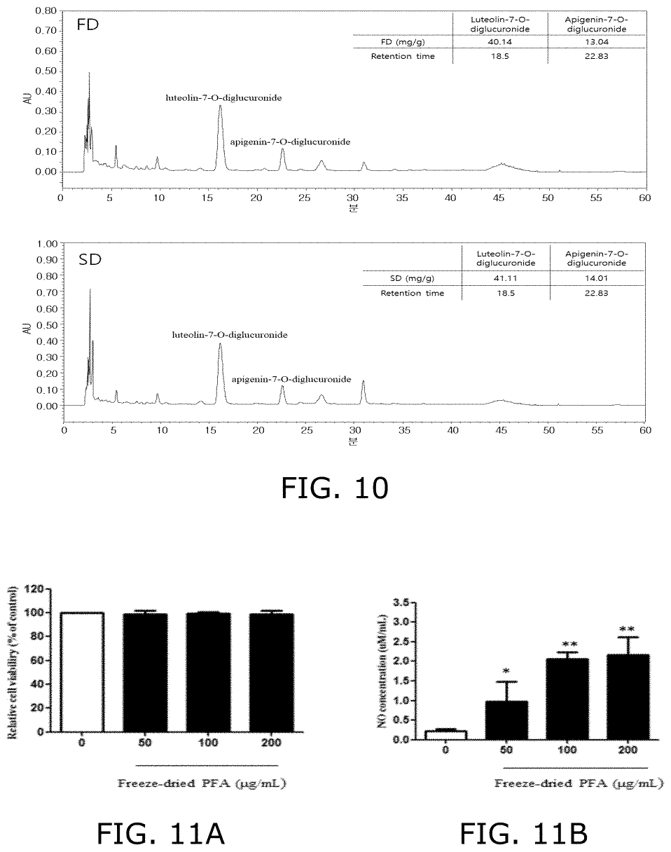

FIG. 10 illustrates the equivalence of luteolin-7-O-diglucuronide and apigenin-7-diglucuronide compounds according to a freeze-drying and spray-drying method of the hot water extract of Perilla frutescens (L.) Britton var. acuta (Thunb.) Kudo leaves. Here, FD represents freeze-dried, and SD represents spray-dried.

3 kg of Perilla frutescens (L.) Britton var. acuta (Thunb.) Kudo was washed with distilled water, added with 30 L of distilled water, and then heated and extracted at 100° C. for 3 hours with an electric kettle. The extracted solution was filtered through a 400 mesh filter cloth and then concentrated under reduced pressure with a vacuum rotary concentrator. Half of the concentrated hot water extract was freeze-dried using a freeze dryer, and the remaining hot water extract was spray-dried with a spray dryer. The luteolin-7-O-diglucuronide contents in the freeze-dried product and the spray-dried product of the hot water extract of Perilla frutescens (L.) Britton var. acuta (Thunb.) Kudo leaves were 40.14 mg/g and 41.11 mg/g, respectively, and the apigenin-7-O-diglucuronide contents were 13.04 mg/g and 14.01 mg/g, respectively, to confirm the equivalence.

3. Isolation of Ciliary Muscle from Rat Eyes and Cell Incubation

To conduct a ciliary muscle cell experiment of a Perilla frutescens (L.) Britton var. acuta (Thunb.) Kudo leaf extract containing luteolin-7-O-diglucuronide and apigenin-7-O-diglucuronide compounds as main ingredients, the ciliary muscle was isolated from the eye of a 3 to 4-week-old Sprague-Dawley rat. That is, the isolated eye was cut in half and the corneal portion was placed in a 15 mL centrifuge tube added with a papain solution and reacted at 37° C. for 90 minutes. The cell suspension was transferred to a new 15 mL centrifuge tube and centrifuged at room temperature (300×g, 5 minutes). After removing the supernatant, the cells were immediately incubated in a DMEM/F-12 (Invitrogen-Gibco, Grand Island, N.Y., USA) medium.

4. Cell Viability and NO Measurement of Ciliary Muscle Isolated from Rat of Perilla frutescens (L.) Britton var. acuta (Thunb.) Kudo Leaf Extract

Ciliary muscle cells (rCSMCs) isolated from rat's eyes were dispensed into a 96 well plate at 1×10⁴ cells/well using a medium containing 10% FBS and incubated for 3 days. After the incubation of 3 days, the Perilla frutescens (L.) Britton var. acuta (Thunb.) Kudo leaf extract containing luteolin-7-O-diglucuronide and apigenin-7-O-diglucuronide compounds as main ingredients was added at concentrations of 50, 100 and 200 μg/mL and reacted in a 37° C. CO₂ incubator for 24 hours. After the reaction, 100 μL of a WST-1 solution was added to each well, reacted at 37° C., and then measured using a microplate reader at a wavelength of 450 nm using the method provided by a manufacturer.

Meanwhile, in NO measurement, ciliary muscle cells (rCSMCs) isolated from rat's eyes were dispensed into a 96 well plate at 5×10⁴ cells/well using a medium containing 10% FBS and incubated for 3 days. After the incubation of 3 days, the Perilla frutescens (L.) Britton var. acuta (Thunb.) Kudo leaf extract containing luteolin-7-O-diglucuronide and apigenin-7-O-diglucuronide compounds as main ingredients was added at concentrations of 50, 100 and 200 μg/mL and reacted in a 37° C. CO₂ incubator for 24 hours. The NO measurement for the supernatant was measured using the Griess reaction.

FIGS. 11A-11B illustrate cytotoxicity and NO content of ciliary muscle cells (rCSMCs) isolated from rat's eyes (SD rat) for the freeze-dried product of the Perilla frutescens (L.) Britton var. acuta (Thunb.) Kudo leaf extract, respectively. 50, 100, and 200 μg/mL of the Perilla frutescens (L.) Britton var. acuta (Thunb.) Kudo leaf extract increased the NO content in a concentration-dependent manner without cytotoxicity.

5. Measurement of Contents of cAMP and cGMP through Ciliary Muscle Cells Isolated from Rat of Perilla frutescens (L.) Britton var. acuta (Thunb.) Kudo Leaf Extract

Ciliary muscle cells (rCSMCs) isolated from rat's eyes were dispensed into 6 wells at 5×10⁵ cells/well and stabilized for one day. After stabilization, 3-isobuytyl-1-methylxanthine (1 mM) was pre-treated for 10 minutes, and then a Perilla frutescens (L.) Britton var. acuta (Thunb.) Kudo leaf extract was reacted for 15 minutes. The contents of cGMP and cAMP of the Perilla frutescens (L.) Britton var. acuta (Thunb.) Kudo extract were measured using an ELISA kit.

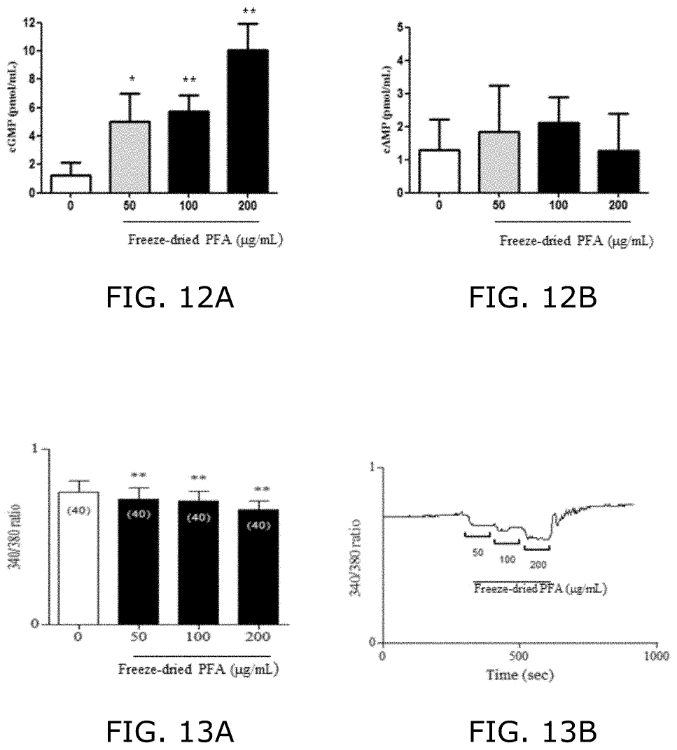

FIGS. 12A-12B illustrate changes in contents of cGMP and cAMP of ciliary muscle cells (rCSMCs) isolated from rat's eyes for the freeze-dried product of the Perilla frutescens (L.) Britton var. acuta (Thunb.) Kudo leaf extract, respectively. 50, 100 and 200 μg/mL of the Perilla frutescens (L.) Britton var. acuta (Thunb.) Kudo leaf extract increased the cGMP content associated with the relaxation of the ciliary muscle in a concentration-dependent manner, while the Perilla frutescens (L.) Britton var. acuta (Thunb.) Kudo leaf extract had no effect on a change in cAMP content.

6. Measurement of [Ca²⁺]i Content through Ciliary Muscle Cells Isolated from Rat of Perilla frutescens (L.) Britton var. acuta (Thunb.) Kudo Leaf Extract

A calcium ion-sensitive fluorescent material, an acetoxymethyl-ester form, fura-2/AM (fura-2/AM; Molecular probes, Eugene, Oreg.) was used as a calcium ion labeling material. The cells were treated with 5 μM fura-2/AM and 0.001% F127 in a HEPES buffer while blocking the light, and reacted at room temperature for 60 minutes. The cells were washed several times with the HEPES buffer and flowed with the buffer for 5 minutes to be stabilized. Hot water extracts (50, 100 and 200 μg/mL) of Perilla frutescens (L.) Britton var. acuta (Thunb.) Kudo were sequentially treated for 100 seconds by concentration. After stabilizing the cells for 5 minutes in the same manner, the hot water extracts of Perilla frutescens (L.) Britton var. acuta (Thunb.) Kudo were treated with one concentration for 100 seconds.

At this time, all buffers and chemicals were treated by a perfusion device by gravity. Light from a lamp was selectively exposed to the cells with wavelengths of 340 nm and 380 nm through a computer control wheel. Photos were taken at 340 nm and 380 nm every 2 seconds, and emitter fluorescence light passing through a 515 nm long-pass filter passed through a cooled CCD camera and obtained a ratio value of 340 nm/380 nm by a digital fluorescence analyzer.

FIGS. 13A-13B illustrate a change in ca²⁺ concentration of ciliary muscle cells (rCSMCs) isolated from rat's eyes for the Perilla frutescens (L.) Britton var. acuta (Thunb.) Kudo leaf extract. FIG. 13A illustrates an effect of the freeze-dried product of the Perilla frutescens (L.) Britton var. acuta (Thunb.) Kudo extract on the [Ca²⁺]i content of ciliary muscle cells isolated from an SD rat, and FIG. 13B illustrates an amount of change in [Ca²⁺]i content of ciliary muscle cells isolated from an SD rat in the freeze-dried product of the Perilla frutescens (L.) Britton var. acuta (Thunb.) Kudo extract. 50, 100 and 200 μg/mL of the Perilla frutescens (L.) Britton var. acuta (Thunb.) Kudo leaf extract decreased the [Ca²⁺]i concentration content associated with the contraction of the ciliary muscle in a concentration-dependent manner.

7. Measurement of cGMP Content through Animal Experiment of Perilla frutescens (L.) Britton var.acuta (Thunb.) Kudo Leaf Extract

As an experimental animal, a 5 to 6-week-old male SD rat (Samtaco) of 180 to 200 g was used. During the entire experiment period, solid feed and water were supplied to be freely ingested, and the rat was reared under conditions of a temperature of 23±3° C., humidity of 50±20%, and a 12-hour light and dark cycle. The experimental animal was used for the experiment after adapted for 1 week in the breeding room. All experimental procedures were performed in accordance with the IACU guidelines and the guidelines for the management and use of experimental animals of the Natural Resources Research Center of Jeonnam Biological Industry Promotion Agency. Animal experiment groups were classified into a normal group which ingested only distilled water for 3 days and did not receive light on the last day, a control group which ingested only distilled water for 3 days and then irradiated light for 15 minutes on the last day, and four groups which irradiated light, ingested a Perilla frutescens (L.) Britton var. acuta (Thunb.) Kudo extract group (100, 200 mg/kg) for 3 days and then irradiated light for 15 minutes on the last day, and in each group, 5 animals were assigned. After the animals were sacrificed, the eyes were extracted from the SD rat and then immediately washed with PBS. The washed eyes were homogenized and then centrifuged to take a supernatant, and its content was measured using a cGMP ELISA kit.

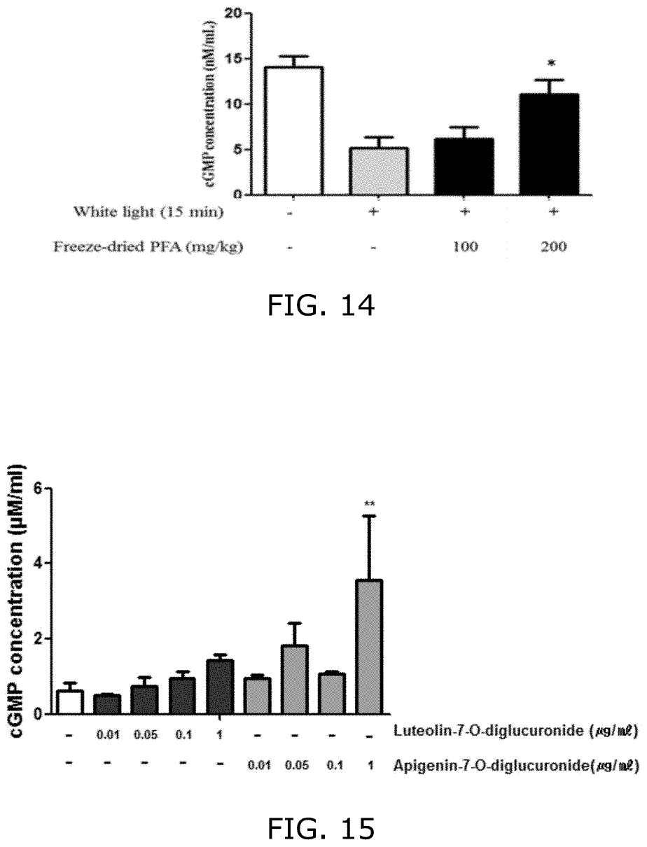

FIG. 14 illustrates a result of measuring a change in cGMP content of rat's eyes after orally administering 100 and 200 mg/kg of the Perilla frutescens (L.) Britton var. acuta (Thunb.) Kudo leaf extract for 3 days after inducing the fatigue by irradiating light to the rat's eyes. It was confirmed that the Perilla frutescens (L.) Britton var. acuta (Thunb.) Kudo leaf extract (100 and 200 mg/kg) increased significantly the cGMP content in the group ingested at a concentration of 200 mg/kg as compared with the control group irradiated with only light to have an effect on relaxation of the eye ciliary muscle.

8. Measurement of cGMP Content of luteolin-7-O-diglucuronide and apigenin-7-O-diglucuronide Compounds Isolated from Perilla frutescens (L.) Britton var.acuta (Thunb.) Kudo Leaf Extract

Ciliary muscle cells (rCSMCs) isolated from rat's eyes were dispensed into 6 wells at 5×10⁵ cells/well and stabilized for one day. After stabilization, 3-isobuytyl-1-methylxanthine (1 mM) was pre-treated for 10 minutes, and then luteolin-7-O-diglucuronide and apigenin-7-O-diglucuronide compounds isolated from the Perilla frutescens (L.) Britton var. acuta (Thunb.) Kudo leaf extract were reacted for 15 minutes at concentrations of 0.01, 0.05, 0.1, and 1 μg/mL. The cGMP contents of these compounds were measured using an ELISA kit.

FIG. 15 illustrates changes in cGMP content of ciliary muscle cells (rCSMCs) isolated from rat's eyes for the Perilla frutescens (L.) Britton var. acuta (Thunb.) Kudo leaf extract. The luteolin-7-O-diglucuronide and apigenin-7-O-diglucuronide compounds isolated from Perilla frutescens (L.) Britton var. acuta (Thunb.) Kudo leaf extract (0.01, 0.05, 0.1, and 1 μg/mL) were confirmed as ingredients of increasing the cGMP content associated with the relaxation of the ciliary muscle. Therefore, it could be seen that the luteolin-7-O-diglucuronide and apigenin-7-O-diglucuronide ingredients among the compounds contained in the Perilla frutescens (L.) Britton var. acuta (Thunb.) Kudo leaf extract affected the eye fatigue effect.

### EXAMPLE 3

### Human Application Test of Eye Control

1. Human Application Test Method

In this study, clinical trials were conducted with the approval of the Medical Device Institutional Review Board (IRB License Number: DSGOH-033) affiliated with D University.

The subjects were screened for a total of 35 applicants for men and women adults over 18 to under 60 who voluntarily received written consent after receiving the explanation of this study. Persons with no congenital or chronic diseases, no pathological symptoms or findings as a result of internal medical examination, blood tests and vital signs test results within a normal range, and an equivalent spherical refractive error of −3.00 D or more were subjected to a screening test, and as a result, persons selected as the final study subject were a total of 30 persons. In this study, the Perilla frutescens (L.) Britton var. acuta (Thunb.) Kudo extract and a placebo control were ingested for 1 week, and a short-range work (VDT) was performed for 2 hours before the last ingestion.

2. Near Point Accommodation Test

While correcting a distant refractive error, the near point accommodation was performed by covering one eye at a distance of 40 cm using a ‘Push-up’ method, and then looking at the number of a near indication clearly, and then measuring the distance of a first blurring point state while pulling close to the uncovered eyes of the subjects. After the right and left eyes were examined, both eyes were examined, and the average value repetitively measured three times was used.

Table 1 illustrates an amount of change in near point accommodation before and after ingestion in a Perilla frutescens (L.) Britton var. acuta (Thunb.) Kudo leaf extract group and a placebo group. The results of comparing the change in the average near point accommodation after performing a visual short-range work for 2 hours before and after ingestion in a hot water extract group of Perilla frutescens (L.) Britton var. acuta (Thunb.) Kudo and a placebo group are as follows. In the right eye of the hot water extract group of Perilla frutescens (L.) Britton var. acuta (Thunb.) Kudo, the near point accommodation decreased statistically significantly from 8.68±2.98 cm before ingestion to 7.83±3.08 cm after ingestion (p<0.001). In the left eye, the near point accommodation decreased significantly from 8.38±3.13 cm before ingestion to 7.67±3.21 cm after ingestion (p<0.001). In the both eyes, the near point accommodation decreased statistically significantly before and after ingestion from 7.96±2.97 cm before ingestion to 7.27±3.25 cm after ingestion (p<0.001).

In the right eye of the placebo group, the near point accommodation increased statistically significantly from 8.90±2.60 cm before ingestion to 9.63±2.40 cm after ingestion (p<0.001). In the left eye, the near point accommodation increased significantly from 8.60±2.49 cm before ingestion to 9.43±2.42 cm after ingestion (p<0.001), and in the both eyes, the near point accommodation increased statistically significantly before and after ingestion from 9.00±2.45 cm before ingestion to 9.67±2.48 cm after ingestion (p<0.001).

TABLE 1
Amounts of change in near point accommodation before and
after ingestion of Perilla frutescens (L.) Britton var.
acuta (Thunb.) Kudo leaf extract group and placebo group

| | | near point accommodation (cm) | | | | |
|---|---|---|---|---|---|---|
| | | Before | After | Change | t-test | p-value |
| A | Right eye | 8.68 ± 2.98 | 7.83 ± 3.08 | −0.85 ± 1.32 | t = −3.53 | p = 0.00* |
| | Left eye | 8.38 ± 3.13 | 7.67 ± 3.21 | −0.71 ± 1.21 | t = −3.20 | p = 0.00* |
| | Binocular | 7.96 ± 2.97 | 7.27 ± 3.25 | −0.69 ± 1.46 | t = −2.60 | p = 0.01* |
| B | Right eye | 8.90 ± 2.60 | 9.63 ± 2.40 | +0.73 ± 0.79 | t = 5.12 | p = 0.00* |
| | Left eye | 8.60 ± 2.49 | 9.43 ± 2.42 | +0.83 ± 1.12 | t = 4.09 | p = 0.00* |
| | Binocular | 9.00 ± 2.45 | 9.67 ± 2.48 | +0.67 ± 0.84 | t = 4.33 | p = 0.00* |

Unit: cm,
*P < 0.05
A: Perilla frutescens (L.)Britton var. acuta (Thunb.)Kudo extract group
B: Placebo control group

### INDUSTRIAL APPLICABILITY

Luteolin-7-O-diglucuronide and apigenin-7-O-diglucuronide compounds contained in a Perilla frutescens (L.) Britton var. acuta (Thunb.) Kudo leaf extract which is a Korean natural resource are used as active ingredients to be usefully used as a pharmaceutical composition and a health functional food composition with a vision improvement function by alleviating eye fatigue including these compounds capable of being safely used without side effects even if ingesting the compounds for a long time. By confirming the equivalence of the compounds of freeze-dried and spray-dried products, it may be expected to reduce manufacturing production costs and to replace imports and increase farm income through industrialization.

What is claimed is:

1. A method of preventing and alleviating eye fatigue in a human in need thereof, comprising: administering to the human in need thereof an effective amount of a pharmaceutical composition, the pharmaceutical composition containing, as active ingredients, luteolin-7-O-diglucuronide and apigenin-7-O-diglucuronide compounds obtained from a Perilla frutescens (L.) Britton var. acuta (Thunb.) Kudo leaf extract.

2. The method of claim 1, wherein the luteolin-7-O-diglucuronide and apigenin-7-O-diglucuronide compounds are isolated and purified by freeze-drying or spray-drying the Perilla frutescens (L.) Britton var. acuta (Thunb.) Kudo leaf extract.

3. The method of claim 1, wherein the luteolin-7-O-diglucuronide and apigenin-7-O-diglucuronide compounds are isolated and purified from a water extract of the Perilla frutescens (L.) Britton var. acuta (Thunb.) Kudo leaves.

4. The method of claim 3, wherein the luteolin-7-O-diglucuronide and apigenin-7-O-diglucuronide compounds are isolated and purified by adding the water extract of the Perilla frutescens (L.) Britton var. acuta (Thunb.) Kudo to a Diaion HP-20 resin column.

5. The method of claim 1, wherein the luteolin-7-O-diglucuronide and apigenin-7-O-diglucuronide compounds alleviate a tension in a ciliary muscle that regulates a focus of an eye caused by VDT syndrome, eye fatigue caused by a short-range work, and aging.

6. The method of claim 5, wherein the luteolin-7-O-diglucuronide and apigenin-7-O-diglucuronide compounds have activities of increasing cGMP contents of ciliary muscle cells.

7. The method of claim 1, wherein the pharmaceutical composition is formed of a formulation selected from tablets, capsules, and liquids.
